(19) United States
(12) Patent Application Publication
(10) Pub. No.: US 2005/0085433 A1
(43) Pub. Date: Apr. 21, 2005

(54) Cellular vaccines comprising adjuvants

(76) Inventors: Claudia Breidenstein, Neu-Esting (DE); John Nieland, Stockdorf (DE)

Correspondence Address: CLARK & ELBING LLP, 101 FEDERAL STREET, BOSTON, MA, 02110, US

(21) Appl. No.: 10/494,716

(22) PCT Filed: Nov. 8, 2002
(86) PCT No.: PCT/EP02/12527

Related U.S. Application Data
(60) Provisional application No. 60/332,497, filed on Nov. 9, 2001.

(51) Int. Cl. A61K 48/00; A61K 39/00

(52) U.S. Cl. 514/44; 424/277.1

(57) ABSTRACT

The invention relates to a composition for vaccination against tumors containing at least one tumor cell, which expresses at least one cytikine, chemokine and/or a con-stimulating molecule and an effective quantity of at least one adjuvant. The invention also relates to the used of a composition of this type for a producing a medicament for the treatment or prevention of tumors.

The present invention relates to cellular vaccines for use in tumor therapy.

Activating the endogenous immune system for the purpose of treating and preventing tumors is a promising approach in modern cancer therapy.

The prior art discloses, inter alia, autologous and allogenic vaccines for the purpose of activating the endogenous immune system (Pardoll D. M., (1998) Nat. Med. 4 (5 Suppl): 525-31; Wolchock J. D. and Livingston P. O., (2001) Lancet Ocol. 2 (4): 205-11; Schadendorf D. et al., (2000) Immunol. Lett. 15; 74 (1): 67-74).

In the case of autologous vaccines, cells from the patient's own tumor are used for producing the vaccine. In this connection, the tumor cells are removed from the body, genetically modified, where appropriate, and made proliferation-incompetent, for example by irradiation, before they are administered to the patient once again. The aim is for immune cells, in particular cytotoxic T cells and helper T cells, to recognize the cells which have been administered and, in this way, to build an immune response which can then also be directed against the tumor.

An alternative to autologous vaccination is what is termed allogenic immunization, i.e. immunizing with cells which are not derived from the same patient. Consequently, the vaccine cells differ from the endogenous cells of the patient since they as a rule do not possess the identical transplantation antigens (MHC genes).

The MHC complex on the surface of cells is of particular importance for developing the specific immune response since peptides are presented in the MHC complex, with these peptides then being recognized by T cells which are specific for them. In this regard, there are two classes of MHC complexes, i.e. class I and class II. MHC-I complexes are expressed on virtually all nucleated vertebrate cells while MHC-II complexes are only present on antigen-presenting cells.

When a specific immune response is developed, a T cell recognizes, by way of its T cell receptor, the MHC complex together with the presented peptide of an antigen and is thereby stimulated to develop an immune response. In this connection, cytotoxic T cells (CTLs) bind to MHC-I complexes and are as a result stimulated to proliferate (clonal selection), while T helper cells bind to MHC-II complexes, likewise resulting in the proliferation of a T cell clone.

However, binding of the T cell receptor to the MHC complex is not usually sufficient for developing a specific immune response. Additional so-called costimulatory molecules are required, with these molecules amplifying the signal exchange between the T cell and the MHC-bearing cell.

The class I MHC complexes are of particular importance for inducing an immune response against tumor cells since the latter present, in their MHC-I complexes, peptides which are found (almost) exclusively on tumor cells, i.e. what are termed tumor antigens, or peptides which are derived from these antigens. It is known in the prior art that the recognition, by particular T cells, of peptides which are derived from tumor antigens and which are presented by MHC class I molecules brings about the proliferation of cytotoxic T lymphocytes (also termed cytotoxic T cells) which are in turn able to destroy tumor cells (Janeway C. et al., (1999) in: Immunobiology; Current Biology Publications, 551-554).

The correct amount of help from T cells (T-cell help) is required for cytotoxic T lymphocytes (CTLS) and antigen-presenting cells to be activated efficiently. This help can be provided, in particular, by Th1 cells and also by Th2 cells. In this connection, Th1 cells principally stimulate a CTL response via IL-12 and IFN-gamma, while Th2 cells promote a B cell response via IL-4 and IL-10. Antigen-presenting cells activate CTLs by means of what is termed cross-priming. If this cross-priming does not take place to a sufficient extent, CTLs which are required for recognizing and eliminating tumor cells are only activated incompletely.

In the case of allogenic vaccination, one (or more) established tumor cell line(s) is/are as a rule used for vaccinating the patient (see WO 97/24132).

Although some degree of immune reaction is elicited in the patient's body simply by administering an allogenic tumor cell line, this immune reaction is as a rule insufficient for controlling the patient's own tumor. For this reason, a variety of attempts have been made in the prior art to elicit an amplification of the immune response by genetically manipulating the tumor cell line which is administered. For example, the prior art (see WO 97/24132) discloses that an amplification of the immune response can be achieved by administering a genetically modified tumor cell which expresses GM-CSF.

All in all, the prior art discloses a large number of allogenic and autologous vaccines which comprise genetically modified tumor cells (Pardoll D. M., (1998) Nat. Med. 4 (5 Suppl): 525-31; Wolchock J. D. and Livingston P. O., (2001) Lancel Ocol. 2 (4): 205-11; Schadendorf D. et al., (2000) Immunol. Lett. 15; 74 (1): 67-74).

Despite this large number of potential vaccines, the prior art does not disclose any vaccine which achieves a satisfactory effect when used in a patient. A disadvantage shared in common by all the vaccines disclosed in the prior art is that the immune response which is induced in the patient is as a rule too weak to effectively combat the patient's own tumor.

The object of the present invention is therefore to provide an improved vaccine in order to efficiently activate the immune system of the host, in order to combat the growing tumor or prevent the development of a tumor.

According to the invention, the object is achieved by means of a composition for vaccinating against tumors, comprising at least one tumor cell, which is expressing at least one cytokine, chemokine and/or a costimulatory molecule, and an effective quantity of at least one adjuvant.

Surprisingly, within the context of the present invention, adjuvants have been found which can be used to efficiently activate the immune system of the tumor patient and thereby combat the growing tumor or prevent the development of a tumor.

In particular, it has been demonstrated that the activity of a cellular vaccine, both in an autologous situation and in an allogenic situation, can be improved by adding CpG oligonucleotide. Furthermore, the window of time within which a vaccination with a cellular vaccine is effective is increased by combining the vaccine with an adjuvant according to the invention. For example, mice in the final stage of a tumor disease still exhibited a response to the cellular vaccination if the vaccine comprised an adjuvant.

The effects of cellular vaccines which expressed transgenes, such as cytokines, chemokines and/or costimulatory molecules, in combination with an adjuvant, such as CpG oligonucleotide, were investigated within the context of the present invention. It was demonstrated, surprisingly, that the presence of a costimulatory molecule, cytokine or chemokine augmented the effect of a vaccine containing an adjuvant, such as CpG, synergically. This is the case, for example, with regard to the expression of a costimulatory molecule such as B7.2 or a cytokine/chemokine such as GM-CSF. In particular, the expression of B7.2 results in the vaccination having a surprisingly large effect when an adjuvant such as CpG has been added to the vaccine. The combination of both a cytokine/chemokine such as GM-CSF and a costimulatory molecule such as. B7.2 also resulted in a surprisingly large effect.

The present invention consequently relates to a composition for vaccinating against tumors, comprising at least one tumor cell, which is expressing at least one cytokine, chemokine and/or costimulatory molecule, and an effective quantity of at least one adjuvant.

Within the context of the present invention, the following definitions are of general importance:

The term “cytokine” is a general designation for a large group of soluble proteins and peptides which function, in nanomolar to picomolar concentrations, as humoral regulators. Under normal or pathological conditions, these regulators modulate the functional activities of individual cells or tissues. In addition, they directly mediate interactions between cells and regulate processes which take place in the extracellular environment.

“Chemokines” are a subgroup of the cytokines. They are relatively small proteins or peptides which, inter alia, have a chemotactic effect on cells.

Within the context of the present invention, a “costimulatory molecule” is a molecule which amplifies the signal exchange between a T cell and an MHC-bearing cell.

Within the context of the present invention, an “adjuvant” is a substance which amplifies the immunogenic (sensitizing) effect of an antigen.

According to the invention, “an effective quantity” of adjuvant denotes a quantity which measurably extends the period of survival of the treated experimental subject as compared with that of a treated experimental subject to whom the tumor cell was administered on its own, or which significantly increases a response in an in-vitro immunoassay.

Within the context of the present invention, a “vaccination against tumors” preferably means that a patient is vaccinated with one of the compositions according to the invention and this thereby treats a tumor, or prevents a tumor, in the patient.

The invention also relates to a composition which comprises at least one tumor cell, which is expressing at least one cytokine, chemokine and/or costimulatory molecule, and an effective quantity of at least one adjuvant.

The following preferred embodiments apply for both compositions according to the invention.

According to a preferred embodiment of the invention, the tumor cell is derived from a pretumor, from a tumor or from a metastasis.

The term “tumor” denotes at least one cell or cell mass in the form of a tissue neoformation, in particular in the form of a spontaneous, autonomous and irreversible excess growth, which is more or less disinhibited, of endogenous tissue, which growth is as a rule associated with the more or less pronounced loss of specific cell and tissue functions. This cell or cell mass is not effectively inhibited, in regard to its growth, by itself or by the regulatory mechanisms of the host organism, e.g. melanoma or carcinoma.

The term “pretumor” denotes at least one cell or cell mass as defined under the term tumor; in contrast to the tumor, however, this cell or cell mass is inhibited, in regard to its growth, by itself or by the regulatory mechanisms of the host organism (e.g. grade 1 cervical intraepithelial neolepsy (CIN1), CIN2 and CIN3).

The term “metastasis” denotes the dissemination of tumor cells and the establishment of secondary regions of the tumor growth. Malignant cells have the ability to metastasize.

According to another preferred embodiment, the tumor cell can be autologous or allogenic with respect to the vaccinated patient. If the vaccination is carried out in an autologous situation, this means that the tumor cell is injected once again into the same patient from whom it was originally derived; the vaccine and the tumor to be treated consequently exhibit the same MHC haplotype. Carrying out the vaccination in an allogenic situation means that the tumor cell which is used for the vaccination is derived from a different patient and consequently as a rule does not possess MHC genes which are identical to those of the endogenous cells of the patient.

According to another preferred embodiment, the tumor cell can be derived from many different types of tumor, for example from a melanoma, ovarian cancer, breast cancer, colon carcinoma, leukemia, lymphoma, renal carcinoma, lung carcinoma, prostate cancer, cervical cancer and/or brain tumor.

Whereas certain tumor cells, such as leukemia cells or lymphoma cells, themselves express particular cytokines and/or chemokines, such as IL-2 or MCP1, or costimulatory molecules, such as B7.1, B7.2, CD40 or CD70, other tumor cells have, in a preferred embodiment, to be genetically modified so that they express one or more molecules from the group comprising cytokines, chemokines and/or costimulatory molecules. Methods for transducing cells are described in the literature, for example in U.S. Pat. No. 6,171,597.

According to a preferred embodiment of this invention, the cytokine/chemokine is selected from the group consisting of GM-CSF, G-CSF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IFN-alpha, IFN-beta, IFN-gamma, Flt3 L, Flt3, TNF-alpha, RANTES, MIP1-alpha, MIP1-beta, MIP1-gamma, MIP1-delta, MIP2, MIP2-alpha, MIP2-beta, MIP3-alpha, MIP3-beta, MIP4, MIP5, MCP1, MCP1-beta, MCP2, MCP3, MCP4, MCP5, MCP6, 6cykine, Dcck1 and DCDF, with GM-CSF, RANTES and/or MIP1-alpha being particularly preferred embodiments.

According to another embodiment of this invention, the costimulatory molecule is selected from the group comprising B7.1,.B7.2, CD40, LIGHT, Ox40, 4.1.BB, I cos, I cos L, SLAM, ICAM-1, LFA-3, B7.3, CD70, HSA (heat stable antigen), CD84, CD7, B7 RP-1 L, MAdCAM-1, VCAM-1, CS-1, CD82, CD30, CD120a, CD120b and TNFR-RP, with B7.1 and B7.2 being particularly preferred embodiments.

According to another embodiment of this invention, these expressed cytokines, chemokines and/or costimulatory molecules are mutated. These mutations include, but are not restricted to, point mutations, deletions or fusions with other peptides or proteins.

Adjuvants according to the invention are preferably those which are suitable for shifting the ratio between the Th2 immune response and the Th1 immune response in favor of the Th1 response.

These adjuvants contrast with other adjuvants whose aim is that of Th2 activation. For example, Her2neu antibodies are used for treating breast cancer, or antiidiotypic antibodies are used in the case of T cell or B cell lymphomas/leukemias, with these antibodies leading to activation of the Th2 response.

As mentioned above, an adequate CTL response is of particular importance for combating tumors and for preventing their development in the patient. A CTL response is in turn particularly helped by a Th1 response, which means the correct ratio between the Th1 immune response and the Th2 immune response is necessary for achieving this aim, i.e. a preferential activation of CTLs.

Without being restricted to the following theory, the limited immune response which is observed in tumor patients can be explained in the following way: in most tumor patients, the ratio between the Th1 immune response and the Th2 immune response is shifted toward the Th2 response, particularly in the case of patients possessing tumors in the final stage (Nieland J D et al. (1998) J Immunother 21, 4, 317-22). There are two mechanisms which lead to this problem: in the first place, all Th2 cells carry an IL-4 receptor which, when it is occupied, increases the resistance of the Th2 cells to Fas-induced apoptosis.

While it is not possible to measure any direct increase in IL4 and IL10 levels in tumor patients, the ratio shifts in favor of IL10 and IL4, was compared with Th1 cytokines, such as IFN, IL2 and TNFα, whose levels frequently fall in tumor patients, such that the immune response is shifted toward the Th2 response.

Furthermore, the altered redox potential in tumor patients leads to an increase in the number of macrophages, which reduce the number of Th1 cells and increase the number of Th2 cells. For this reason, cytokines which are relevant for a CTL response, and which specifically stimulate a Th1 response, such as IFN-gamma and IL-12, and also molecules such as CD40L, are not adequately expressed.

Examples of adjuvants which can be used to efficiently activate the immune response, which is limited in tumor patients, in particular, in order to combat the tumor or prevent its development are Toll-like receptor agonists, such as CpG oligonucleotide, lipopolysaccharides or Calmette-Guerin bacillus cell wall skeleton (CWS), and also superantigens and agents which inhibit the CTLA-4 signal effect.

The term “agonist” denotes a physiological substance or a pharmaceutical which triggers an effect by occupying a membrane receptor.

The term “Toll-like receptor” denotes receptors which exhibit homology with the Toll receptors which are known from Drosophila. These receptors are seen as being mediators of the danger signal (Matzinger P., (2002) Ann. N. Y. Acad. Sci. 961: 341-2; Matzinger P., (1994) Annu. Rev. Immunol. 12: 991-1045). They react to bacterial or viral signals, such as bacterial DNA, CpG motifs, double-stranded RNA and bacterial or viral proteins.

CpGs are synthetic DNA fragments which contain what are termed the “CpG motifs” which are found in bacterial DNA. Bacterial DNA has the property of possessing a large number of unmethylated CpG motifs. They are present at a frequency of 1/16 in bacteria, as compared with 1/50-60 in mammalian DNA, where they are suppressed (Chen Y et al. (2001) Int Immunol 13, 1013-20).

Within the context of the present invention, “CpG” denotes one or more oligonucleotide(s) containing at least one CpG motif.

CpGs imitate the stimulatory effect of bacterial DNA. As a factor of innate immunity, they influence both the nonspecific and the specific immune responses. It is known from the literature that CpG intervenes in several steps of the immune response. CpG interacts with Toll-like receptors on various immune cells such as macrophages, dendritic cells and NK cells. The normal ligands for Toll-like receptors are LPS and other PAMPs (pathogen-associated molecular patterns) (Wagner H (2001) Immunity 14, 499-502). As a consequence of the binding of ligands to Toll-like receptors, Th1 cytokines such as IFN-gamma and IL-12 are strongly upregulated. Inflammation-promoting (proinflammatory) cytokines, including TNF-alpha (tumor necrosis factor-alpha), IL-6 and IFN type I, have the same fate. In addition, NK cells are activated to secrete IFN-gamma, and their lytic activity is augmented (Chen et al. 2001). A polarization of the T helper response from Th2 toward Th1 is initiated (Krieg AM et al. (1999) Pharmacol Ther 84, 2, 113-20; Kranzer K et al. (2000) Immunology 99, 2, 170-8). This leads to activation of immature dendritic cells by CD40L on Th cells. T helper 1 cells are able to stimulate a response involving specific CTLs. As discussed above, this appears, without being restricted to this theory, to be of particular value when vaccinating against cancer since, in cancer cases, the responses of the T helper cells are frequently shifted in the direction of the Th2-mediated immune response, particularly in the case of patients possessing tumors in the final stage. Furthermore, CpG also activates antigen-presenting cells, resulting in the sensitization (priming) of CTLs being improved (Hacker H et al. (2000) J Exp Med 192, 4, 595-600), Kranzer et al. see above).

Synthetic CpG is able to imitate the immunostimulatory effect of bacterial DNA. It therefore appears to be a good adjuvant; surprisingly, it also appears to be a good adjuvant in attempts to vaccinate against tumors.

According to another preferred embodiment of the invention, the adjuvant is therefore an agonist of a Toll-like receptor.

According to another preferred embodiment, the adjuvant is selected from the group consisting of CpG oligonucleotides, LPS and BCG-CWS.

There is a series of different possible CpG motifs which stimulate the various immune cells to differing extents. We use a special motif which Coley Pharmaceuticals made available to use within the context of our collaboration and is configured so as to stimulate Th1 and NK cells optimally (obtained from Coley Pharmaceuticals, #M426).

U.S. Pat. No. 6,218,371 discloses that it is possible to observe a synergic effect when immunostimulatory CpG oligonucleotides and immunopotentiating cytokines, such as GM-CSF, are combined. On the other hand, this publication does not describe the combination with cellular vaccines.

Another embodiment of this invention consists in the CpG oligonucleotide being an oligonucleotide which has a sequence which contains at least the following formula:

5′ X₁CGX₂ 3′

with the oligonucleotide containing at least 8 nucleotides, with C being unmethylated and with X₁ and X₂ being nucleotides.

Within the context of the present invention, a “nucleotide” includes, for example, adenosine, cytidine, guanosine, thymidine or uridine, or modified forms thereof.

According to another embodiment of the invention, the G in the oligonucleotide sequence 5′ X₁CGX₂ 3′ is additionally unmethylated.

According to another embodiment of the invention, the CpG oligonucleotide is an oligonucleotide which has a sequence which contains at least the following formula:

5′ N₁X₁CGX₂N₂ 3′

with at least one nucleotide separating consecutive CpGs and with X₁ being adenine, guanine or thymine, and with X₂ being cytosine, adenine or thymine, and with N being any arbitrary nucleotide and with N₁ and N₂ being nucleic acid sequences which are in each case composed of approximately 0-25 nucleotides. According to a particularly preferred embodiment, N₁ and N₂ of the nucleic acid do not contain any CCGG tetramer (quadramer) or do not contain more than one CCG or CGG trimer.

Another embodiment of this invention consists in the CpG oligonucleotide being an isolated oligpnucleotide which has a sequence which contains at least the following formula:

5′ N₁X₁X₂CGX₃X₄N₂ 3′

with at least one nucleotide separating consecutive CpGs and with X₁X₂ being selected from the group consisting of GpT, GpA, ApA, GpG and ApT and with X₃X₄ being selected from the group consisting of TpT, CpT, TpC, CpC and ApT, and with N being any arbitrary nucleotide and with N₁ and N₂ being nucleic acid sequences which are in each case composed of approximately 0-25 nucleotides.

According to another preferred embodiment, N₁ and N₂ of the nucleic acid do not contain any CCGG tetramer (quadramer) or do not contain more than one CCG or CGG trimer.

Another embodiment of this invention consists in the CpG oligonucleotide having a nucleic acid sequence in which N₁ and N₂ do not contain any CCGG tetramer (quadramer) or do not contain more than one CCG or CGG trimer.

Within the context of the present invention, a “CCGG tetramer” denotes an oligonucleotide which consists of the nucleotide sequence CCGG and a “CCG or, respectively, CGG trimer” denotes an oligonucleotide which consists of the nucleotide sequence CCG or, respectively, CGG.

According to another embodiment of the invention, the CpG oligonucleotide is an oligonucleotide which has the sequence:

5′ TCN₁TX₁X₂CGX₃X₄ 3′

with at least one nucleotide separating consecutive CpGs and with X₁X₂ being selected from the group consisting of GpT, GpA, ApA, GpG and ApT, and with X₃X₄ being selected from the group consisting of TpT, CpT, TpC, CpC and ApT, and with N being any arbitrary nucleotide and with N₁ and N₂ being nucleic acid sequences which are in each case composed of approximately 0-25 nucleotides.

According to another embodiment of this invention, the CpG oligonucleotides are coupled to the surface of the cell. The Qligonucleotides can be bound covalently to the surface, e.g. by means of crosslinkings or, for example, by means of an interaction between a cell membrane protein and the CpG oligonucleotide. One possibility is to express an IgM immunoglobulin which is specific for the respective CpG oligonucleotide and to incubate the tumor cells with the respective CpG oligonucleotides before the cells are injected into the patient. CpG-polylysine complexes could also be coupled to the surface of the tumor cell. Bispecific antibodies can also be used to couple CpG or other adjuvants to a membrane protein belonging to the tumor cell.

The term “oligonucleotide” is used interchangeably and denotes multiple nucleotides (i.e. molecules containing a sugar (e.g. ribose or deoxyribose) which are linked to a phosphate group and an exchangeable organic base which is either a substituted pyrimidine (e.g. cytosine (C), thymine (T) or uracil (U)), or a substituted purine (e.g. adenine (A) or guanine (G)). As used herein, the term refers both to oligoribonucleotides and oligodeoxyribonucleotides. The term is also intended to encompass polynucleosides (i.e. a polynucleotide minus the phosphate) and any other polymer which contains organic bases. While nucleic acid molecules can be obtained from existing sources of nucleic acids, (e.g. genomic or cDNA), they are preferably synthetic (e.g. produced by means of oligonucleotide synthesis). While the entire CpG oligonucleotide can be completely or partially unmethylated, at least the C in the 5′ CG 3′ has to be unmethylated. The CpG oligonucleotide according to the invention preferably contains X₁X₂ which is selected from the group consisting of GpT, GpG, GpA and. ApA, and X₃X₄ which is selected from the group consisting of TpT, CpT and GpT. In order to facilitate uptake into cells, the length of the CpG-containing oligonucleotides is preferably in the range from 8 to 30 bases. However, nucleic acids of any arbitrary size greater than 8 nucleotides (even many kb in length) are able to induce an immune response according to the invention as long as a sufficiently large number of immunostimulatory motifs is present, since larger nucleic acids are broken down within cells to form oligonucleotides. Preferred synthetic oligonucleotides do not contain any CCGG tetramer (quadramer), or do not contain more than one CCG or CGG trimer, at or close to the 5′ and/or 3′ ends. Preference is also given to stabilized oligonucleotides, where the oligonucleotide contains a modification of the phosphate backbone, as discussed in more detail below. The modification can, for example, be a phosphorothioate or phosphorodithioate modification. The modification of the phosphate backbone is preferably effected at the 5′ end of the nucleic acid, for example at the first two nucleotides of the 5′ end of the oligonucleotide. The modification of the phosphate backbone can also be effected at the 3′ end of the nucleic acid, for example at the last 5 nucleotides of the 3′ end of the nucleic acid. As an alternative, the oligonucleotide can be completely or partially modified.

The CpG oligonucleotide is preferably within the range between 8 and 100, and particularly preferably between 8 and 30, nucleotides in size. As an alternative, CpG oligonucleotides can be produced on a large scale in plasmids and broken down to form oligonucleotides.

The CpG oligonucleotide and at least one immunopotentiating cytokine can be administered directly to the experimental subject being treated or can be administered together with a nucleic acid delivery complex. “Nucleic acid/cytokine delivery complex” is intended to denote a nucleic acid molecule and/or a cytokine which is associated with (e.g. ionically or covalently bound to, or encapsulated within) a targeting agent (e.g. a molecule which results in a relatively high-affinity binding to the target cell (e.g. surfaces of dendritic cells and/or an increase in the uptake by target cells)). Examples of nucleic acid/cytokine delivery complexes comprise nucleic acid/cytokines which are associated with: a sterol (e.g. cholesterol), a lipid (e.g. a cationic lipid, virosome or liposome) or a target cell-specific binding agent (e.g. a ligand which is recognized by a target cell-specific receptor). Preference is given to the complexes being sufficiently stable in vivo so as to ensure that significant decoupling prior to internalization by the target cell is prevented.

Nevertheless, preference is given to it being possible for the complex to be cleaved, under suitable conditions, within the cell such that the nucleic acid/cytokine is released in functional form.

The CpG oligonucleotide can be an oligonucleotide which contains palindromic sequences. “Palindromic sequence” is intended to denote an inverted repeat (i.e. a sequence such as ABCDEE′D′C′B′A′, where A and A′ are bases which are able to form the customary Watson-Crick base pairs). In vivo, such sequences can form double-stranded structures. In one embodiment, the CpG oligonucleotide contains a palindromic sequence. In this connection, a palindromic sequence refers to a palindrome in which the CpG is part of the palindrome and is preferably the center of the palindrome. In another embodiment, the CpG oligonucleotide is free from a palindrome. A CpG oligonucleotide which is free from a palindrome is one in which the CpG dinucleotide is not part of a palindrome. Such an oligonucleotide can contain a palindrome, with the CpG not being part of the palindrome.

The CpG oligonucleotide can be a stabilized nucleic acid molecule. A “stabilized nucleic acid molecule” is intended to denote a nucleic acid molecule which is relatively resistant to breakdown in vivo (e.g. brought about by an exonuclease or an endonuclease). Stabilization can be a function of the length or of the secondary structure. Unmethylated CpG bligonucleotides which are from several 10 kb to several 100 kb in length are relatively resistant to breakdown in vivo. The secondary structure can stabilize shorter CpG oligonucleotides and increase their effect. If, for example, the 3′ end of an oligonucleotide exhibits self complementarity with a region which is located further upstream, such that the oligonucleotide can fold back and form a type of stem loop structure, the oligonucleotide is then stabilized and therefore exhibits more activity.

Stabilized oligonucleotides of the present invention which are preferred have a modified backbone. It has been shown that the modification of the oligonucleotide backbone increases the activity of the CpG oligonucleotide when the latter is administered in vivo. CpG constructs which exhibited at least two phosphorothioate linkages at the 5′ end of the oligonucleotide and several phosphorothioate linkages, preferably 5 such linkages, at the 3′ end brought about maximal activity and protected the oligonucleotide from breakdown by intracellular exonucleases and endonucleases. Other modified oligonucleotides include phosphodiester-modified oligonucleotides, combinations of phosphodiester and phosphorothioate oligo-nucleotides, methyl phosphonate, methyl phosphorothioate and phosphorodithioate, and combinations thereof. Each of these combinations, and their particular effect on immune cells, is discussed in more detail in U.S. Pat. No. 6,207,646 and U.S. Pat. No. 6,239,116, and the entire content of the latter two publications is hereby incorporated into this application by this reference. It is assumed that these modified oligonucleotides are able to exhibit greater stimulatory activity on account of their increased resistance to nucleases, on account of an increase in cellular uptake, on account of an increase in protein binding and/or on account of changed intracellular locations.

Both phosphorothioate oligonucleotides and phosphodiester oligonucleotides which contain CpG motifs are active in APCs such as dendritic cells. However, based on the concentration which is required in order to induce CpG-specific effects, the CpG oligonucleotides which possess a nuclease-resistant phosphorothioate backbone are more active (2 μg/ml in the case of the phosphorothioates versus a total quantity of 90 μg/ml in the case of phosphodiesters).

Other stabilized oligonucleotides include: nonionic DNA analogs, such as alkyl phosphates and aryl phosphates (in which the charged phosphonate oxygen is replaced by an alkyl or aryl group), phosphodiesters and alkyl phosphotriesters in which the charged oxygen group is alkylated. Oligonucleotides which contain diol, such as tetraethylene glycol or hexaethylene glycol, at one or other end or at both ends are known to be essentially resistant to breakdown by nucleases.

Other adjuvants according to the invention which act as Toll-like receptor agonists are lipopolysaccharides (LPSs), or components thereof, such as the lipid A moiety or the polysaccharide or oligosaccharide moiety. LPSs are the principal outer membrane components of virtually all Gram-negative bacteria and are known to have powerful stimulatory effects on the immune system. LPSs consist of a polysaccharide or oligosaccharide region which is anchored in the outer bacterial membrane by lipid A. The specific, cellular recognition of the LPS/lipid A is mediated by the joint extracellular interaction of the LPS-binding protein, the membrane-bound or soluble form of CD14 and the Toll-like receptor 4*MD2 complex. This leads to rapid activation of an intracellular signal network which has strong homology with the IL-1 and IL-8 signal cascade (Alexander C and Rietschel ET (2001) J Endotoxin Res 7, 3, 167-202).

According to another embodiment of the invention, therefore, the adjuvant is LPS.

According to another embodiment, the adjuvant is derived from Calmette-Guerin bacillus cell wall skeleton (BCG-CWS). BCG-CWS is known to be a ligand of the Toll-like receptors 2 and 4 and can induce differentiation of immune cells (Matsumoto M et al (2001) Int Immunopharmacol 1, 8, 1559-69).

According to another embodiment of the invention, the adjuvant is a superantigen. Superantigens are antigens which bind directly to T cell receptors and MHC molecules and activate the T cells directly. Superantigens are also known to be able to have an adjuvant effect (see, for example, Okamoto S et al (2001) Infect. Immun. 69, 11, 6633-42). Examples of known superantigens are Staphylococcus aureus enterotoxins A, B, C, D and E (SEA, SEB, SEC, SED and SEE), Staphylococcal aureus toxic shock syndrome toxin 1 (TSST-1), staphylococcal exfoliating toxin and streptococcal pyrogenic exotoxins.

According to another embodiment of the invention, the adjuvant is an agent which inhibits the CTLA-4 signal effect.

CTLA-4 (cytotoxic T lymphocyte-associated antigen 4) is a receptor which, after having been activated, retards the immune response since its functions as an antagonist to CD28. While it is expressed in low copy number by activated T cells, it binds to B7 with an affinity which is approx 20-fold higher than that of the latter's actual receptor CD28. It is known that a soluble form of the extracellular domain of CTLA-4 binds B7 and suppresses T cell-dependent antibody immune responses in vivo.

In this case, the agent can, for example, be antibodies or antibody fragments which bind specifically to the extracellular domain of CTLA-4 and inhibit its signal effect. The skilled person is familiar with the generation and/or screening of such antibodies and/or antibody fragments (see, for example, WO 0032231). Other agents which are suitable for binding CTLA-4 and inhibiting its signaling are small organic molecules, peptide analogs or soluble T cell receptors (see WO 9720574).

The invention also relates to the use of a composition which comprises at least one tumor cell, which is expressing at least one cytokine, chemokine and/or costimulation molecule, and an effective quantity of at least one adjuvant for producing a pharmaceutical for treating or preventing tumors. That which has been said above applies to the tumor cell, the cytokine, chemokine and/or costimulatory molecule and to the adjuvant.

The invention also relates to a process for producing a pharmaceutical for treating or preventing tumors, with at least one tumor cell, which is expressing at least one cytokine, chemokine and/or costimulatory molecule, and an effective quantity of at least one adjuvant being mixed.

The invention also relates to a process for treating or preventing tumors in which an effective quantity of tumor cells, which are expressed in at least one cytokine, chemokine and/or costimulatory molecule, and an effective quantity of at least one adjuvant, are administered to a patient. That which has been said above applies to the tumor cell, the cytokine, chemokine and/or costimulatory molecule and to the adjuvant.

According to a preferred embodiment, the tumor cell which is expressing at least one cytokine, chemokine and/or costimulatory molecule is produced by transducing it with recombinant adenoassociated virus (AAV). AAV vectors were prepared as described in WO 00/47757.

The term “transduction with recombinant adenoassociated virus (AAV)” is understood as meaning that the gene(s) for a cytokine, chemokine and/or costimulatory molecule is/are introduced into the cell using one or more recombinant AAVs and is/are expressed as a consequence thereof. The preparation of suitable recombinant AAVs is well-known to the skilled person (see, for example, WO 00/47757). The AAV vectors which were used within the context of this invention were prepared using the methods described in WO 00/47757.

According to another preferred embodiment, the adjuvant is added to the cell suspension. The cells and adjuvants are then mixed with each other. Other auxiliary substances and additives are added where appropriate.

### EXAMPLE

### Example 1

1. Materials

1. Cell Lines and Animals:

Female C3H/He mice aged 6-7 weeks were obtained from Harlan, Borchen, Germany. The melanoma cell line K-1735-M2 was kindly provided by Dr. Souberbielle (King's College, London) and Prof. I. J. Fidler (University of Texas M. D. Anderson Cancer Center, Houston, USA). The known mouse melanoma cell line B16F10 was also used.

CpG oligodinucleotides were made available as the result of a collaboration with Coley Pharmaceuticals Group™.

2. Methods

1. Generating HEL-Expressing Tumor-Cells:

For an allogenic vaccination scheme:

Two stable HEL-expressing cell lines, which were generated for the allogenic vaccination experiment, were used: B16-HEL-61 (H2-b) and K-1735-HEL-48 (H2-k).

The expression vector pcDNA3neo-HEL was cloned for the purpose of generating stable transfectants of the B16F10 and K-173,5 melanoma cells. To do this, the HEL gene was excised from the vector pcDNA1-HEL and ligated into the expression vector pcDNA3neo, which carries a gene for resistance to neomycin for the selection. B16F10 and K-1735 cells were transfected in a 15 cm cell culture dish using Lipofectamine®. Positive cells were selected using G418-containing selection medium (800 μg/ml). After 2-3 weeks, individual clones were picked and expanded. The clones were tested by RT-PCR and Western blotting for expression of the transgene. The two clones giving the best expression rate were selected for the vaccination experiments.

RT—PCR:

The RNA was prepared using 2-5×10⁶ cells, QIAshredder columns (#79654) and the QIAgen® RNeasy kit (#74104). DNA (e.g. episomal plasmid DNA) was removed using RNAse-free DNAse (#776785, Roche®). RNA was transcribed into cDNA using the Gene Amp RNA PCR core kit (Perkin Elmer®, #N808-0143). PCRs for HEL and β-actin were carried out using the QIAgen Taq Mastermix kit (#1007 544) and the following primers:

| | |
|---|---|
| HEL-up | (5′ -AGG TCT TTG CTA ATC TTG GTG C-3′) |
| HEL-down | (5′ -GGC AGC CTC TGA TCC ACG-3′) |
| mu β- | (5′ -GAT CCT GAC CGA GCG TGG CTA C-3′) |
| actin-up | |
| mu β- | (5′ -CAA CGT CAC ACT TCA TGA TGG AAT TG-3′) |
| actin- | |
| down | |

The fragments which were obtained were the HEL fragment (430 bp) and the β-actin-fragment (290 bp).

Western Blot:

Cells were lysed with cell lysis buffer. Lysates were run on 12% polyacrylamide gels using DTT-containing loading buffer. Chick egg lysozyme (Sigma®, #L4631) was used as the standard. The transfer to nitrocellulose membranes was carried out using a semidry transfer system. Blocking and labeling with antibodies were carried out in a 5% solution (in Tris-buffered saline solution, 0.01% Tween (TBST)) of dry skimmed milk.

Antibody: biotinylated anti-HEL 1:200 (RDI, #RDI-lyszym-BT)

Streptavidin-HRP 1:5000 (Sigma®, #S-5512)

Super Signal (Pierce®, #34080) was used as the substrate. X-ray films were exposed for from 30 seconds to one hour.

2. Generating B7.2-Expressing and GM-CSF-Expressing K1735 (-HEL) and B16F10-HEL Vaccination Cells:

K1735 and K-1735-HEL cells, which are expressed in murine B7.2 or GM-CSF, or both molecules, were prepared by transducing with recombinant adenoassociated virus (AAV). AAV vectors were prepared as described in WO 00/47757.

B16-HEL cells, which cannot be transduced efficiently with recombinant AAV, were transfected with Polyfect (QIAgen, #301107) so as to express the two molecules B7.2 -and/or GM-CSF transiently. While the rates at which GM-CSF was expressed were comparable to those in K1735-HEL cells, the rates at which B7.2 was expressed were somewhat lower.

The vaccination cells were irradiated and stored in liquid nitrogen.

In order to prepare cells for administration to the mice, they were thawed, washed three times with PBS and adjusted, in PBS, to a cell count of 3×10⁵ cells per dose.

3. Detecting the Expression of GM-CSF and B7.2:

The OptEIA mouse GM-CSF set enzyme-coupled immunoassay (ELISA) kit from Pharmingen (San Diego, USA) was used to detect secreted GM-CSF, after 48 hours, in the supernatant from transduced or transfected cells. The antibody GL1 (Pharmingen) was used to detect the expression of B7.2 by flow cytometry.

4. Using CpG Oligonucleotides as Adjuvants:

In the case of groups which were inoculated with adjuvant, CpG was added to the cell suspension or PBS at a concentration of 10 μg per dose.

5. Analyzing the Lung Metastases:

Mice were killed by dislocating the neck, after which the lungs were dissected out, weighed and fixed. Bouin's reagent was used for the C3H mouse lungs (reference to: Current protocols in Immunology). The number of metastases was counted using a dissecting microscope.

6. Preparing Spleen Cells/T Cells

The spleens of the vaccinated mice were removed when the animals were dissected and stored in medium until they were subjected to further processing. In order to obtain a suspension of single cells, the spleens were disrupted using a cell strainer (70 μl/Nunc®). The cells were washed once and then purified from macrophages by being passed through nylon wool. The extracted T cells were restimulated once a week with irradiated, autologous tumor cells. Rat spleen ConA sup (T stim™ culture supplement, Collaborative Biomedical Products, #354115) was added at a concentration of 1-3% for the purpose of improving growth.

7. ⁵¹Cr-Release Test:

5 days after the restimulation, the T cell cultures were harvested, washed and plated out, as triplet samples, on 96-well round-bottomed plates at a cell count of 1.8×10⁵, 6×10⁴, 2×10⁴ and 6.7×10³ cells per well. Live target cells were labeled, at 37° C. for one hour, with ⁵¹chromium, washed four times and added so as to obtain a final ratio of effector to target cell of 90:1, 30:1, 10:1 and 3:1. In order to block NK lysis, unlabeled YAC-1 cells were added to the target cells at a ratio of from 1:5 to 1:10. After incubating for 5 hours, the supernatants were collected and transferred to LUMA plates. On the following day, the dried plates were counted in a β counter (Packard). Specific lysis was calculated using the following formula:
% specific lysis=(test lysis−spontaneous lysis)/(maximum lysis−spontaneous lysis)* 100

### Example 2

1. Therapeutic, Autologous Vaccination Against K1735 Melanoma With and Without CpG:

| | | | |
|---|---|---|---|
| Species/ | Test | | |
| strain: | compounds: | Purpose: | Design: |
| C3H/He | K-1735 | Prevention of | 6 × 10⁴ unmodified |
| mouse | cells | the formation | K1735 cells were |
| strain | transduced | of tumor | administered i.v. to |
| (H-2k) | with rAAV- | metastases | C3H/He mice. |
| Age, sex: | B7.2 and | following | Beginning on day 4, |
| 6-7-week- | GM-CSF | vaccination | 7 or 11, PBS +/− CpG |
| old | (H-2k) +/− CpG | with B7.2/GM- | or genetically |
| female | PBS +/− CpG | CSF vaccines | modified and |
| mice | Dose, | +/− CpG as | irradiated variants |
| Body | route: | compared with | of syngenic K1735 |
| weight: | s.c., 3 × 10⁵ | control | cells +/− CpG were |
| 30 g | cells, | vaccines. | vaccinated s.c., as |
| TV 20, 24 | 10 μg of | Laboratory: | vaccine, at a dose |
| | CpG per | preclinical | of 3 × 10⁵ cells (twice |
| | dose | development | at an interval of |
| | | division, | 7 days). The |
| | | MediGene AG, | development of lung |
| | | Munich, | metastases was |
| | | BioService, | analyzed 21 days |
| | | Munich, non- | after the challenge |
| | | GLP study | with the tumor. |

Mice which had been vaccinated with K-1735 cells which were coexpressing B7.2GM-CSF developed a lower average lung weight than did animals which had been vaccinated with PBS (206.4±13. 6 mg as compared with 339.5±75.8 mg and 166.80±10.7 mg as compared with 200.75±42.8 mg, respectively). Combining K1735-B7.2-GM-CSF with CpG increased the therapeutic effect. In all groups, a delay in beginning the vaccination reduced the therapeutic effect with the exception of. K1735-B7.2-GM+CpG. In the latter group, comparable results were seen in all the groups, with a certain degree of variation. In TV20, the group which was vaccinated with PBS beginning on days 7 and 11 gave strange results which cannot be explained and which did not recur in subsequent experiments.

Two autologous therapeutic experiments have thus far been carried out in C3H/He mice. The results are summarized in the following table. Experiment TV24 gave a lower tumor burden and, as a result, no clear differences between the groups:

| | | Experiment 1 (TV20) | | | Experiment 2 (TV24) | | |
|---|---|---|---|---|---|---|---|
| | | (see FIGS. 1 and 2) | | | (see FIGS. 3 and 4) | | |
| | | 2× vaccination with 3 × 10⁵ | | | 2× vaccination with 3 × 10⁵ | | |
| | | cells beginning | | | cells beginning on | | |
| | | on day 4, 7 or 11 after | | | day 4, 7 or 11 after challenge | | |
| | | a challenge with | | | with 1 × 10⁵ wt | | |
| | | 1 × 10⁵ wt tumor cells. | | | tumor cells. Analysis on | | |
| | | Analysis on day 21 | | | day 21 following | | |
| | | following tumor induction | | | tumor induction | | |
| | | N = 4/10 per group | | | N = 4/10 per group | | |
| | | | Average | | | Average | |
| | | Animals | number of | | Animals | number | |
| | Vaccination | with | lung | Average lung | with | of lung | Average lung |
| Vaccine | begun on | metastases | metastases | weight in mg | metastases | metastases | weight in mg |
| PBS | day 4 | 4/4 | 111.75 | 339.5 | 4/4 | 54.00 | 200.75 |
| | | | (+/−18.18) | (+/−75.75) | | (+/−14.80) | (+/−42.77) |
| PBS | day 7 | 4/4 | 95.5 | 291.5 | 4/4 | 47.75 | 179.75 |
| | | | (+/−28.91) | (+/−56.28) | | (+/−13.24) | (+/−19.88) |
| PBS | day 11 | 4/4 | 29.5 | 187.75 | 3/3* | 83.33 | 213.00 |
| | | | (+/−5.52) | (+/−22.95) | | (+/−31.71) | (+/−36.09) |
| PBS + CpG | day 4 | 4/4 | 64.75 | 234.25 | 4/4 | 37.75 | 164.25 |
| | | | (+/−23.32) | (+/−27.91) | | (+/−4.52) | (+/−13.24) |
| PBS + CpG | day 7 | 4/4 | 42.75 | 211.25 | 4/4 | 32.50 | 170.00 |
| | | | (+/−16.04) | (+/−19.89) | | (+/−5.63) | (+/−12.37) |
| PBS + CpG | day 11 | 4/4 | 60.00 | 220.0 | 4/4 | 44.75 | 196.75 |
| | | | (+/−13.25) | (+/−15.66) | | (+/−10.63) | (+/−14.74) |
| K1735-B7.2- | day 4 | 10/10 | 63.3 | 206.4 | 4/4 | 25.80 | 166.80 |
| GM | | | (+/−11.90) | (+/−13.58) | | (+/−7.52) | (+/−10.71) |
| K1735-B7.2- | day 7 | 10/10 | 71.4 | 263.0 | 9/9* | 29.56 | 188.00 |
| GM | | | (+/−18.44) | (+/−37.8) | | (+/−4.57) | (+/−12.60) |
| K1735-B7.2- | day 11 | 10/10 | 68.90 | 263.3 | 10/10 | 36.30 | 196.50 |
| GM | | | (+/−13.99) | (+/−33.1) | | (+/−8.84) | (+/−16.78) |
| K1735-B7.2- | day 4 | 10/10 | 18.9 | 173.3 | 10/10 | 28.20 | 174.4 |
| GM + CpG | | | (+/−10.12) | (+/−14.02) | | (+/−11.29) | (+/−17.43) |
| K1735-B7.2- | day 7 | 10/10 | 39.2 | 226.6 | 10/10 | 29.70 | 173.00 |
| GM + CpG | | | (+/−10.56) | (+/−39.1) | | (+/−7.13) | (+/−8.52) |
| K1735-B7.2- | day 11 | 10/10 | 36.6 | 173.2 | 10/10 | 24.50 | 163.60 |
| GM + CpG | | | (+/−7.82) | (+/−9.13) | | (+/−5.84) | (+/−8.52) |

*One fatality, probably not connected with the treatment
Data: mean value and standard error

In two separate experiments (TV26 and TV27), tumor induction was elicited in C3H/He mice by intravenously injecting the mice with 6×10⁴ K-1735 cells on day 0. The vaccination was carried out on days 4 and 11 in 3 groups using 3×10⁵ K-1735 cells. In group 1, the-cells were WT K-1735 cells while, in group 2, they were K-1735 cells which were expressing B7.2, and in group 3 they were K-1735 cells which were -expressing B7.2 together with CpG. On day 21, the animals were sacrificed and the mean lung weight and the number of metastases were determined.

In both experiments, the combination of B7.2 and CpG gave the best therapeutic effect (TV26, see FIG. 5A), with experiment TV27 showing that B7.2 and CpG had a clear synergic effect (FIG. 5B). However, the differences between the groups using B7.2 and B7.2CpG were not significant in experiment TV26 (FIG. 5A), a finding which can be explained by the rather low number of metastases which arose in all the groups in this specific experiment. The combination of CpG and B7.2 therefore exhibits a synergic effect in this mouse model.

In comparison with similar experiments using tumor cells which expressed other cytokines and/or costimulatory molecules, the combination of B7.2 and CpG appeared to achieve one of the most powerful synergic effects which has thus far been observed.

2. Therapeutic Allogenic Vaccination Against K1735-HEL Melanoma With and Without CpG Oligonucleotide:

| | | | |
|---|---|---|---|
| Species/ | Test | | |
| strain: | compounds: | Purpose: | Design: |
| C3H/He | B16F10-HEL | Prevention/ | 1.2 × 10⁵ unmodified |
| mouse | wt cells | retardation of | K1735-HEL cells were |
| strain | and | tumor | administered i.v. to |
| (H-2k) | transfected | dissemination/ | C3H/He mice. 4 and |
| Age, sex: | with: B7.2- | growth by | 11 days later, |
| 6-7-week- | and/or GM- | B7.2/GM-CSF | genetically modified |
| old | CSF-pAAV | vaccines as | and irradiated |
| female | plasmid | compared with | variants of |
| mice | (H-2b) | control | allogenic B16F10-HEL |
| Body | K-1735-HEL | vaccines. | cells and syngenic |
| weight: | transduced | Effect of CpG | K1735-HEL cells were |
| 30 g | with rAAV- | on the | administered s.c. as |
| TV 22 | B7.2/GM-CSF | allogenic | vaccines at a dose |
| | (H2-k) | vaccination. | of 3 × 10⁵ cells. The |
| | Dose, | Laboratory: | development of lung |
| | route: | preclinical | metastases was |
| | s.c., 3 × 10⁵ | development | analyzed 21 days |
| | cells | division, | after the tumor |
| | | MediGene AG, | induction. |
| | | Munich, | |
| | | BioService, | |
| | | Munich, non- | |
| | | GLP study | |

Mice which, after tumor induction using wt tumor cells, were vaccinated with B16F10-HEL cells which were coexpressing B7.2 GM-CSF developed a significantly lower average lung weight than did animals which were inoculated with control wild-type cells (404 mg as compared with 564 mg (FIG. 6A)). The therapeutic effects achieved by B7.2 GM-CSF-expressing autologous and allogenic vaccination cells were comparable. In addition, this suggests that the vaccine exhibits tumor-reducing activity even at a time at which the body already has a growing tumor mass.

An effect which was similar, but less pronounced, was observed when comparing the metastasis nodes in the lungs of treated mice and control animals (FIG. 6B).

Experiment 3 (TV22) (see FIGS. 6A and B)

| | 2× vaccination with 3 × 10⁵ cells on | | |
|---|---|---|---|
| | days 4 and 11 following challenge | | |
| | with 1 × 10⁵ wt tumor cells. Analysis | | |
| | on day 21 after the tumor induction. | | |
| | N = 10 per group | | |
| | | Average | |
| | Animals | number of | Average |
| | possessing | lung | lung weight |
| | metastases | metastases | in mg |
| B16-HEL-wt | 10/10 | 160.00 | 303.5 |
| | | (+/−79.34) | (+/−38.86) |
| B16-HEL-B7.2 | 10/10 | 201.2 | 373.4 |
| | | (+/−72.01) | (+/−47.36) |
| B16-HEL-GM-CSF | 10/10 | 157.7 | 304.3 |
| | | (+/−59.21) | (+/−36.38) |
| B16-HEL-B7.2/GM- | 9/9* | 172.33 | 273.22 |
| CSF | | (+/−54.05) | (+/−30.17) |
| K1835-HEL-B7.2/GM | 10/10 | 169.8 | 255.2 |
| (autologous) | | (+/−69.18) | (+/−29.46) |
| B16-HEL-wt + CpG | 10/10 | 162.9 | 288.9 |
| | | (+/−59.1) | (+/−32.14) |
| B16-HEL-B7.1/GM- | 10/10 | 98.3 | 195.8 |
| CSF + CpG | | (+/−54.7) | (+/−17.54) |

*One fatality, probably not associated with the treatment
Data: mean values and standard errors

3. T Cell Experiments:

In order to test the lytic potential of the T cells derived from vaccinated mice, spleen cells were cultured and restimulated as described in Materials and Methods. A chromium release assay against autologous target cells was carried out as the test system.

The spleen cells derived from in each case 2 animals, which originated from experiment TV20 and which belonged to the following groups, were cultured:

| Vaccine | Commencement of vaccination |
|---|---|
| PBS | 4 |
| PBS + CpG | 4 |
| PBS + CpG | 11 |
| K1735-B7.2-GM-CSF | 4 |
| K1735-B7.2-GM-CSF | 4 |
| K1735-B7.2-GM-CSF + CpG | 4 |
| K1735-B7.2-GM-CSF + CpG | 11 |

It turned out that it was only possible to efficiently expand T cells which were derived from mice which had been vaccinated with K-1735-B7.2-GM-CSF cells with or without CpG. PBS or PBS and CpG did not appear to be a sufficient stimulus for inducing a T cell proliferation which was adequate for long-term expansion in vitro. Following 2-3 rounds of restimulation, the only cell lines which grew well were those shown in FIG. 7.

The cells derived from K-1735-B7.2-GM-CSF(±CpG)-vaccinated mice exhibited lysis of the target cells in the chromium release assay. In this case, the combination of a vaccine and CpG gave the best effects (animal number 79). Up to 70% specific lysis was observed. FIG. 7 shows an example of such a chromium assay.

4. Analysis

In the experiments which we carried out, we attempted to combine the effect of CpG, as adjuvant, with cellular melanoma vaccines which were carrying B7.2 and GM-CSF. The intention was that this would result in a direct activation, by B7.2 on the tumor cell, of naive CD8 T cells and NK cells and, in addition, lead, as the result of the effect of GM-CSF, to the recruitment and maturation of dendritic cells. The latter cells take up and process antigens and present them in the correct context, in order to activate CD8 T cells by way of MHC-I and CD4 T cells by way of MHC-II. In addition, the CpG motif, which we used, activates APCs, NK cells and CD4 Th1 cells, which already, on their own, block Th2 cells and, in addition, activate DCs by expressing CD40L. The intention was that the cooperation of all these factors would lead to an effective immunoactivation which was able to combat an existing tumor.

This potential was tested in an autologous situation (vaccine and tumor sharing the same MHC haplotype). In addition, CpG was also used in an allogenic situation in which no MHC congruence existed. Tumor cell lines which carried HEL (chick egg lysozyme) as the model tumor antigen possessed in common were established for this experiment.

In this series of experiments, the potential of the CpG oligonucleotides, acting as adjuvants, to support a developing immune response, induced by the vaccine, against the tumor was investigated in vaccination studies carried out in a mouse melanoma model.

As tumor model, we used K-1735 cells (H2-k haplotype) in an autologous therapeutic vaccination scheme in C3H/He mice. In order to obtain lung metastases, live tumor cells were injected into the tail vein. The vaccines employed were K-1735 cells, which had been transduced with rAAV-muB7.2-GM-CSF, or PBS, without, or in combination with, CpG. The vaccination was begun on day 4, 7 or 11 following tumor induction (challenge). On day 21 after the tumor induction (challenge), the animals were sacrificed in order to determine the lung weight and the number of lung metastasis nodes.

As an alternative, CpG was used, in one case, in an allogenic situation employing B16F10-HEL (H2-b haplotype) and K-1735-HEL (H2-k haplotype) cells in C3H/He mice. In this case, pAAV-muB7.2-GM-CSF-transfected B16F10-HEL cells were used as a completely allogenic vaccine. Chick egg lysozyme (HEL) is a model antigen which is derived from chick egg white and which is known in the literature to be a good antigen (Calin-Laurens V et al. (1993) Vaccine 11, 9, 974-8; Cavani A et al. (1995) J Immunol 154, 3, 1232-8; Forquet F et al. (1990) Eur J Immunol 20, 2325-32; Schneider S C et al. (20.00) J Immunol 165, 1, 20-3; Thatcher T H et al. (2000) Immunology 99, 2, 235-42). It was used as a model tumor antigen in the allogenic situation. CpG was administered together with B16-HEL wild-type cells or in combination with B7.2-GM-CSF-transfected cells. The transduced/transfected vaccination cells were irradiated before being administered subcutaneously to the animals in order to prevent tumor growth.

CpG augmented the effect of both an autologous vaccine and an allogenic vaccine.

PBS on its own, as well as PBS and CpG, did not have any effect, or only had a slight effect, on metastasis formation. Delaying the vaccination to day 7 or 11 minimized the “vaccination effect”. While vaccination with irradiated tumor cells which were expressing B7.2GM-CSF reduced the number of lung metastases, it did not do this as powerfully as when used in combination with CpG.

Furthermore, in contrast to when vaccinating without CpG, a time delay in regard to beginning the vaccination after administering the live tumor cells (tumor induction) was tolerated when CpG was used. Thus, the lung weight-and the number of metastases were as high when beginning the vaccination on day 4 as when beginning the vaccination on day 11. This consequently widens the time window for achieving successful therapy. This effect was tied to tumor cells which were carrying a transgene also being administered, which meant that the success of the vaccination was antigen-dependent.

CpG also increased the effect of a cellular vaccine in an allogenic situation. In this case, combination with allogenic wild-type cells on their own hardly had any effect whereas combination with B7.2-GM-CSF-expressing cells drastically increased the vaccination effect in comparison to cells without CpG.

The two mouse melanoma models B16F10 and K1735 were used as tumor models which were relevant for comparing the effects of an allogenic vaccine and an autologous vaccine in the case of malignant melanoma. However, the experimental nature of animal tumor models in general, and the heterogeneity between different tumors (mouse versus human, between individual patients) must be borne in mind. These models are more likely to be able to provide qualitative information of a comparative nature, rather than absolute quantitative information, about the therapeutic efficacy in a human situation, when this is taken into account.

The purpose of these experiments was to demonstrate the stimulatory effect of CpG, as an adjuvant, when carrying out a cellular tumor vaccination against a melanoma. According to the above-discussed theory, to which the invention is not to be restricted, the efficacy of the vaccine should be increased when the polarizing effect of CpG in favor of a Th1 response, and the activation of antigen-presenting cells, is efficient.

When using CpG in an autologous situation, it was possible to shift the beginning of the vaccination toward the end of the experiment. In the experiment depicted in FIG. 1, the three K-1735-B7.2-GM-CSF+CpG groups exhibit virtually the same lung weight whereas the mean lung weight increases in the three K-1735-B7.2-GM-CSF groups depending on whether the vaccination was carried out on day 4, 7 or 11 following tumor induction. This shows that the commencement of the vaccination is not so critical when CpG is used as when it is not used. Even with a commencement on day 11, when animals are given their second vaccination three days before the dissection, the tumor burden was not higher than when vaccination commenced on day 4.

This effect appears to apply particularly when the CpG vaccination is combined with antigens and transgenes. This can be seen in the experiment depicted in FIG. 6, in which, while autologous tumor cells together with CpG have a relatively small effect, the same vaccine, but expressing B7.2 and GM-CSF, drastically reduces the tumor burden of the animals. CpG on its own had some effect (see example, item 3). However, in this case, as well as in the case of vaccinating only with B7.2-GM-CSF-expressing cells, the therapeutic effect decreases when there was a time delay in the first vaccination following the tumor induction (challenge). This scenario can be seen in both the experiments which are depicted in FIGS. 1 and 2 as well as in FIGS. 3 and 4. In these cases, the time of the first vaccination appears to be critical, as was demonstrated by van Elsas et al. (1999, J Exp Emd 190, 355-66). According to one possible explanation, to which the invention is not to be restricted, this might be attributable to a downregulation of the CD3 ζ chain in the T cell receptor of the T helper cells, due to their activation, or to a preferential Fas L-induced downregulation of T helper 1 responses.

In the case of a therapeutic vaccination, particularly good effects were induced by a vaccination which commenced on day 4 following the tumor induction (challenge) A later start was less advantageous. The experiments which were performed show that the combination of a vaccine which is expressing B7.2 and GM-CSF together with CpG is able to put an end to this negative correlation. Consequently, the time of the vaccination is not critical in the case of the compositions according to the invention, something which constitutes an important advantage of the present invention.

The T cell experiments which were carried out support the data from the animal experiments using autologous vaccines.

The ⁵¹Cr release test shows that the tumor reduction effects which were achieved in animals which had been vaccinated with PBS and CpG must be due to an innate immunity and cannot be a case of T cell stimulation since no specific lysis was found. Only animals which also received cellular antigen (K-1735-B7.2-GM-CSF) were able to develop a distinct T cell response, something which cannot only be seen by the tumor rejection in vivo but can also be detected in a ⁵¹Cr release assay, thereby verifying the lytic activity of T cells. Since the data on chromium release were recorded 2-3 weeks after the spleen cells had been taken into culture, the cells responsible cannot be NK cells. After 2-3 weeks in culture, most of the NK cells have normally disappeared. In addition, unlabeled YAC-1 cells were added in the chromium release experiments in order to block NK lysis. For this reason, the calculated values for the specific lysis correspond to cytotoxic T cell lysis.

Taken overall, all the experiments support the role of CpG as an adjuvant for autologous and allogenic vaccination for increasing stimulation of specific cytotoxic T cells and inducing rejection of the tumor.

### DESCRIPTION OF THE FIGURES

FIG. 1: Therapeutic (autologous) vaccination with B7.2/GM-CSF and/or CpG (TV20): Mice were sacrificed on day 21 after the challenge and the lung weight was determined on a microbalance. The figure depicts the mean lung weight in mg plotted against the respective experimental formulation. The mean lung weight for a healthy mouse is 140 mg. The numbers given in brackets denote the day of the first vaccination (day 4, 7 or 11).

FIG. 2: Therapeutic (autologous) vaccination with B7.2/GM-CSF and/or CpG (TV20): Mice were sacrificed on day 21 after the challenge, after which the lungs were fixed in a Bound's solution and the lung metastases were determined using a dissecting microscope. The figure depicts the mean number of lung metastases plotted against the respective experimental formulation. The numbers shown in brackets denote the day of the first vaccination (day 4, 7 or 11).

FIG. 3: Therapeutic (autologous) vaccination with B7.2/GM-CSF and/or CpG (TV24): Mice were sacrificed on day 21 after the challenge and the lungs were weighed. The figure depicts the mean lung weight in mg plotted against the respective experimental formulation. The mean lung weight for a healthy mouse is 140 mg. The numbers shown in brackets denote the day for the first vaccination (day 4, 7 or 1l).

FIG. 4: Therapeutic (autologous) vaccination with B7.2/GM-CSF and/or CpG (TV24): Mice were sacrificed on day 21 after the challenge, after which the lungs were fixed in a Bouin's solution and the lung metastases were determined using a dissecting microscope. The figure depicts the mean number of lung metastases plotted against the respective experimental formulation. The numbers given in brackets denote the day for the first vaccination (day 4, 7 or 11).

FIG. 5: Therapeutic vaccination of C3H/He mice with transduced melanoma cells; the figure depicts the mean lung weight in mg plotted against the respective experimental formulation. The mean lung weight for a healthy mouse is 140 mg. FIG. 5A shows experiment TV26 while FIG. 5B shows experiment TV27.

FIG. 6A: Therapeutic (allogenic) vaccination of C3H/He mice with transduced melanoma cells (TV22): Mice were sacrificed on day 21 after the challenge and the lung weights were determined on a microbalance. The figure depicts the mean lung weight in mg plotted against the respective experimental formulation. The mean lung weight for a healthy mouse is 140 mg.

FIG. 6B: Therapeutic (allogenic) vaccination of C3H/He mice with transduced melanoma cells (TV22): Mice were sacrificed on day 21 after the challenge, after which the lungs were fixed in a Bouin's solution and the lungs were determined using a dissecting microscope. The figure depicts the mean number of lung metastases plotted against the respective experimental formulation.

FIG. 7: ⁵¹Cr release assay for spleen cells originating from TV20: Spleen cells derived from animals originating from TV20 were restimulated in vitro with irradiated K-1735-HEL cells. On day 5 after restimulation, the cells were incubated for 4 hours with 51-chromium-labeled target cells (K-1735-HEL) using the effector cell/target cell ratios indicated. Supernatants were measured in a β counter. The figure depicts the % specific lysis plotted against the ratio of effector cells to target cells (E:T).

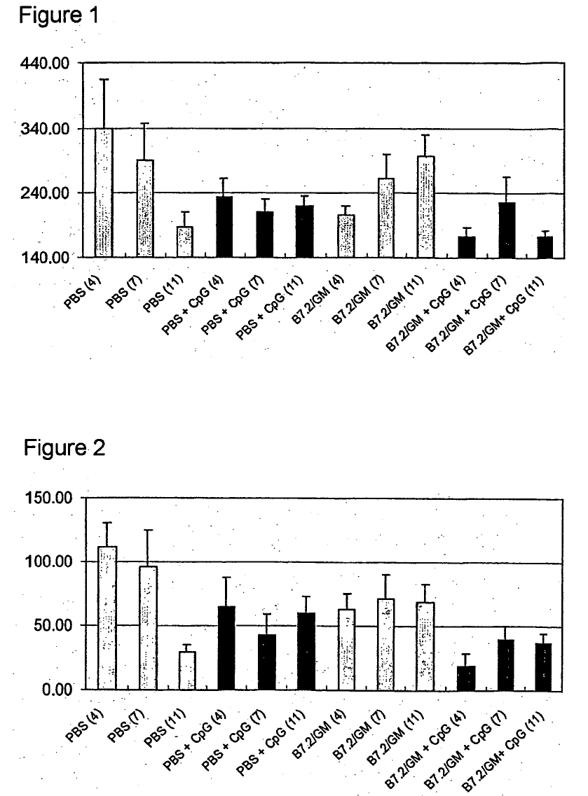

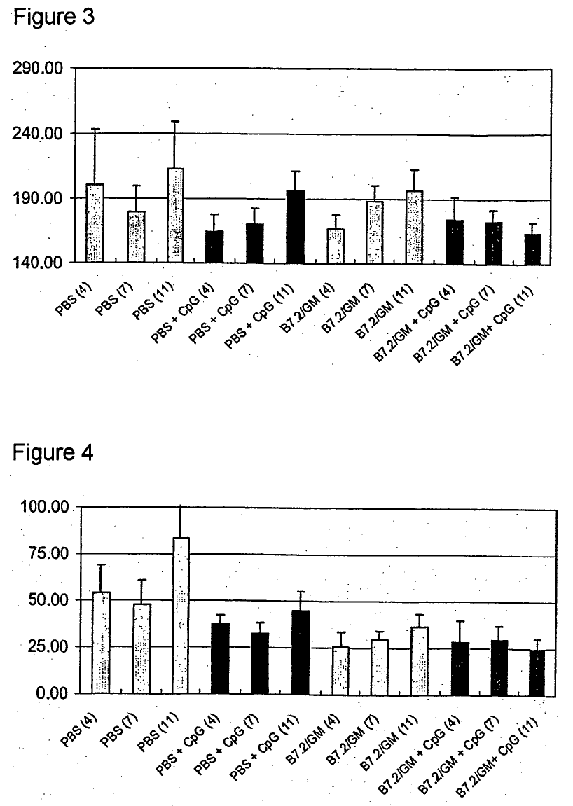

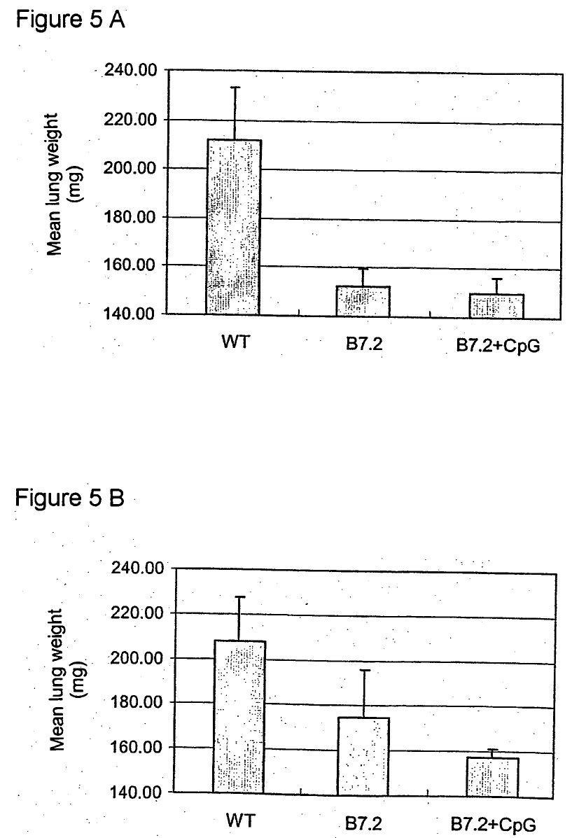

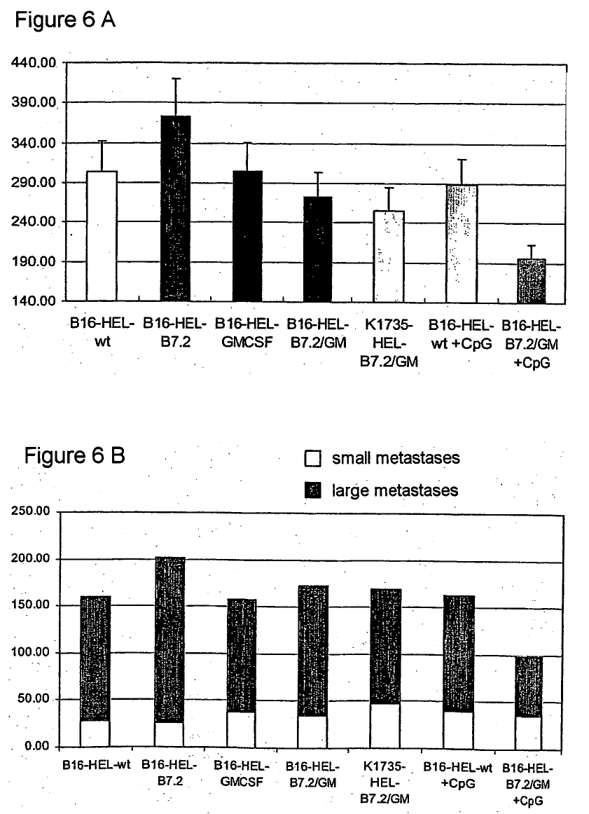

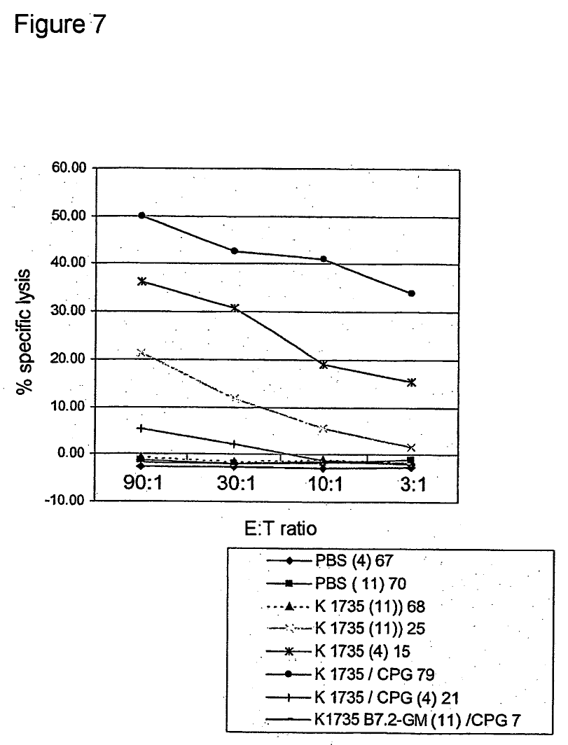

1-28. (canceled)

29. A composition for vaccinating against tumors, comprising at least one tumor cell which is expressing a molecule selecting from the group consisting of at least one cytokine, chemokine and costimulatory molecule; and an effective quantity of at least one adjuvant.

30. A composition comprising at least one tumor cell, which is expressing a molecule selecting from the group consisting of at least one cytokine, chemokine and costimulatory molecule; and an effective quantity of at least one adjuvant.

31. A composition as claimed in claim 29, characterized in that the tumor cell is derived from a tumor selected from the group consisting of a pretumor, a tumor and a metastasis.

32. A composition as claimed in claim 29, characterized in that the tumor cell is autologous or allogenic in regard to the vaccinated patient.

33. A composition as claimed in claim 29, characterized in that the tumor cell is derived from a tumor selected from the group consisting of a melanoma, ovarian cancer, breast cancer, colon carcinoma, leukemia, lymphoma, renal carcinoma, lung carcinoma, prostate cancer, cervical cancer and brain tumor.

34. A composition as claimed in claim 29, characterized in that the tumor cell is modified genetically such that it expresses one or more molecules selected from the group consisting of cytokines, chemokines and costimulatory molecules.

35. A composition as claimed in claim 29, characterized in that the molecule is selected from the group consisting GM-CSF, G-CSF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IFN-alpha, IFN-beta, IFN-gamma, Flt3 L, Flt3, TNF-alpha, RANTES, MIP 1-alpha, MIP 1-beta, MIP 1 -gamma, MIP 1-delta, MIP2, MIP2-alpha, MIP2-beta, MIP3-alpha, MIP3-beta, MIP4, MIP5, MCP1, MCP1-beta, MCP2, MCP3, MCP4, MCP5, MCP6, 6cykine, Dcck1 and DCDF.

36. A composition as claimed in claim 29, characterized in that the costimulatory molecule is selected from the group consisting of B7.1, B7.2, CD40, LIGHT, Ox40, 4.1.BB, Icos, Icos L, SLAM, ICAM-1, LFA-3, B7.3, CD70, HSA, CD84, CD7, B7 RP-1 L, MAdCAM-1, VCAM-1, CS-1, CD82, CD30, CD120a, CD120b and TNFR-RP.

37. A composition as claimed in claims 29, characterized in that the molecule is a mutated molecule, including point mutations, deletions or fusions with other peptides or proteins.

38. A composition as claimed in claim 29, characterized in that the adjuvant is an agonist of a Toll-like receptor.

39. A composition as claimed in claim 29, characterized in that the adjuvant is selected from the group consisting of CpG oligonucleotides, LPS and BCG-CWS.

40. A composition as claimed in claim 29, characterized in that the CpG oligonucleotide has a sequence which contains at least the following formula:
5′ X₁CGX₂ 3′
where the oligonucleotide contains at least 8 nucleotides, with C being unmethylated and with X₁ and X₂ being nucleotides.

41. A composition as claimed in claim 40, characterized in that the G is additionally unmethylated.

42. A composition as claimed in claim 29, characterized in that the CpG oligonucleotide has a sequence which contains at least the following formula:
5′ N₁X₁CGX₂N₂ 3′
where at least one nucleotide separates consecutive CpGs and where X₁ is adenine, guanine or thymine and where X₂ is cytosine, adenine or thymine and where N is any arbitrary nucleotide and where N₁ and N₂ are nucleic acid sequences each of which is composed of approximately 0-25 nucleotides.

43. A composition as claimed in claim 29, characterized in that the CpG oligonucleotide has a sequence which contains at least the following formula:
5′ N₁X₁X₂CGX₃X₄N₂ 3′
where at least one nucleotide separates consecutive CpGs and where X₁X₂ is selected from the group consisting of GpT, GpA, ApA, GpG and ApT and where X₃X₄ is selected from the group consisting of TpT, CpT, TpC, CpC and ApT, and where N is any arbitrary nucleotide and where N₁ and N₂ are nucleic acid sequences each of which is composed of approximately 0-25 nucleotides.

44. A composition as claimed in claim 42 or 43, where N₁ and N₂ of the nucleic acids do not contain any CCGG tetramer (quadramer) or do not contain more than one CCG or CGG trimer.

45. A composition as claimed in claim 29, characterized in that the CpG oligonucleotide has the sequence:
5′ TCN₁TX₁X₂CGX₃X₄ 3′
where at least one nucleotide separates consecutive CpGs and where X₁X₂ is selected from the group consisting of GpT, GpA, ApA, GpG and ApT, and where X₃X₄ is selected from the group consisting of TpT, CpT, TpC, CpC and ApT, and where N is any arbitrary nucleotide and where N₁ and N₂ are nucleic acid sequences each of which is composed of approximately 0-25 nucleotides.

46. A composition as claimed in claim 40, characterized in that the CpG oligonucleotide is coupled to the surface of the cell.

47. A composition as claimed in claim 29, characterized in that the adjuvant is a superantigen.

48. A composition as claimed in claim 29, characterized in that the adjuvant is an agent which inhibits the CTLA-4 signal effect.

49. A method of treating or preventing tumors, said method comprising administering to a patient a composition comprising at least one tumor cell, which is expressing at least one molecule selected from the group consisting of a cytokine, chemokine and costimulatory molecule, and an effective quantity of at least one adjuvant.

50. A process for producing a composition as claimed in claim 29, characterized in that at least one tumor cell, which is expressing at least one molecule selected from the group consisting of a cytokine, chemokine and costimulatory molecule, and an effective quantity of at least one adjuvant, are mixed.

51. The process as claimed in claim 50, characterized in that the tumor cell, is repared by means of transduction with recombinant adenoassociated virus (AAV).

52. The process as claimed in claim 50, characterized in that the adjuvant is added to the cell suspension.
